(19) United States
(12) Plant Patent Application Publication
(10) Pub. No.: US 2016/0192566 P1
(43) Pub. Date: Jun. 30, 2016

(54) CHRYSANTHEMUM PLANT NAMED 'ZANMUGLOW YELLOW'

(50) ;

(71) Applicant: Chrysanthemum Breeders Association Research B.V., Valkenburg ZH (NL)

(72) Inventor: Henricus Cornelius Maria Jacobs, Valkenburg (NL)

(21) Appl. No.: 14/544,365

(22) Filed: Dec. 30, 2014

(51) Int. Cl. A01H 5/00

(52) U.S. Cl. PLT/295

(57) ABSTRACT

A chrysanthemum plant named ‘Zanmuglow Yellow’ characterized by its medium sized blooms with yellow ray florets and prolific branching; natural season flower date September 22 (week 39); blooming for a period of 5 weeks.

### BOTANICAL DESIGNATION

Chrysanthemum×morifolium Ramat.

### CULTIVAR DENOMINATION

‘Zanmuglow Yellow’

### RELATED CULTIVARS

The new plant is related to ‘Zanmuglow’ (U.S. Plant Pat. No. 22,617). ‘Zanmuglow Yellow’ is a mutant of ‘Zanmuglow’.

### BACKGROUND OF THE INVENTION

The present invention relates to a new and distinct cultivar of chrysanthemum plant, botanically known as Chrysanthemum×morifolium Ramat., commercially known as a garden mum, and hereinafter referred to by the cultivar denomination ‘Zanmuglow Yellow’. ‘Zanmuglow Yellow’ is a product of a selection program which had the objective of finding color mutants from existing parent plants. The new plant comprises a natural mutant of the parent chrysanthemum named ‘Zanmuglow’. Plants from the new cultivar ‘Zanmuglow Yellow’ differ from plants of the parent in (1) color of the ray-florets. And (2) Inflorescence size. (1) The color is yellow in the mutant, while it is white in the parent. (2). Inflorescences of the mutant are smaller in size than those of the parent.

The new and distinct cultivar was discovered and selected as a flowering plant by Henricus Cornelius Maria Jacobs on a cultivated field in Rijsenhout, The Netherlands in 2009. The first act of asexual production of ‘Zanmuglow’ was accomplished when vegetative cuttings from the initial selection in 2009 were propagated further in a controlled environment in Rijsenhout, The Netherlands. The new cultivar has been found to retain its distinctive characteristics through successive propagations.

### BRIEF DESCRIPTION OF THE DRAWINGS

The present invention of a new and distinct variety of chrysanthemum (of about 16 weeks) is shown in the accompanying drawings, the color being as nearly true as possible with color photographs of this type.

FIG. 1 shows a plant of the cultivar in full bloom.

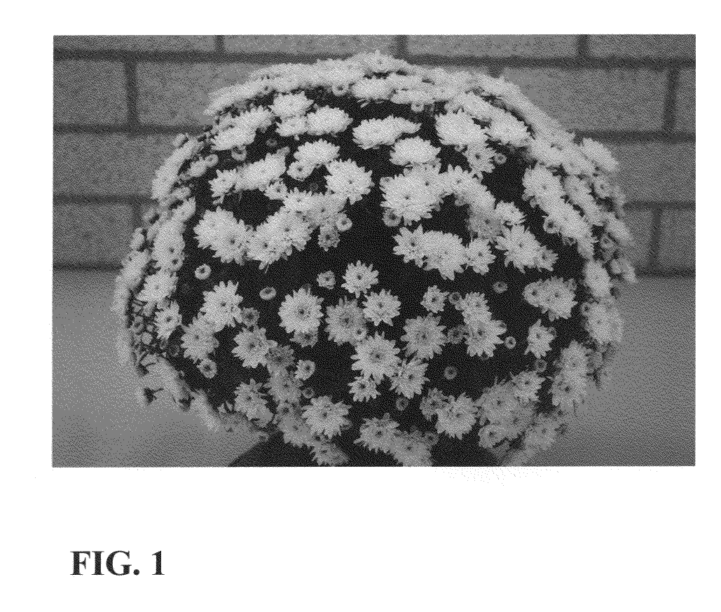

FIG. 2 shows the various stages of bloom of the new cultivar.

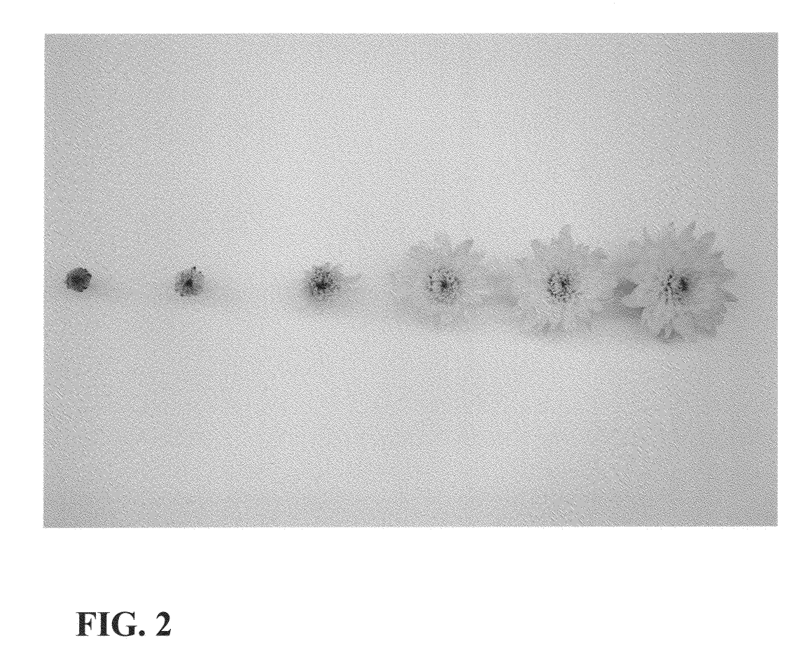

FIG. 3 shows the various stages of foliage of the new cultivar.

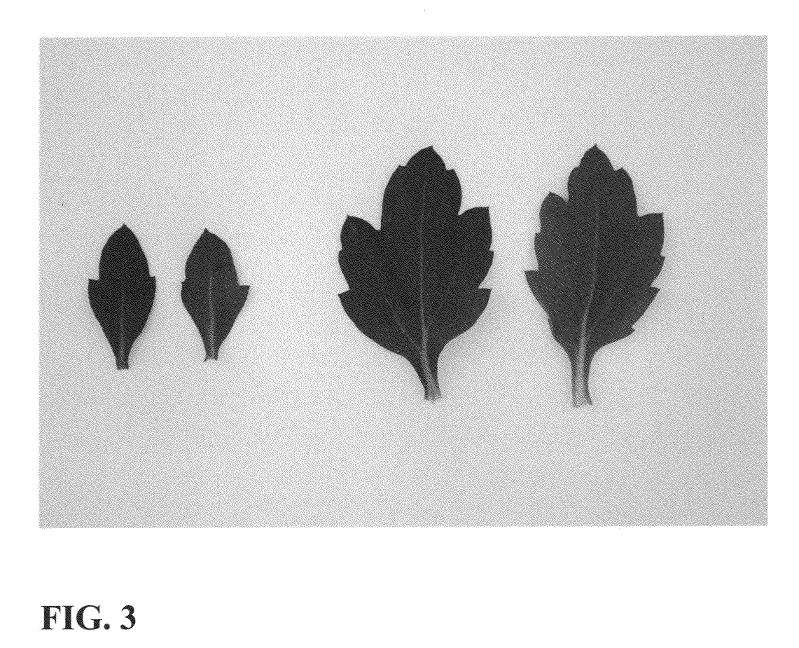

### DESCRIPTION OF THE INVENTION

The observations and measurements were gathered from plants grown out door in Rijsenhout, The Netherlands under natural day length and temperature and planted in week 25 in 2014. The natural blooming date of this crop was September 22 (week 39) . The average height of the plants was 30 cm. No growth retardants were used. No tests were done on disease or insect resistance or susceptibility. No tests were done on cold or drought tolerance. This new variety produces medium sized blooms with yellow ray florets blooming for a period of 5 weeks.

From the cultivars known to inventor the most similar existing cultivar in comparison to ‘Zanmuglow Yellow’ is ‘Gold Crest’ (U.S. Plant Pat. No. 11,606). When ‘Gold Crest’ and ‘Zanmuglow Yellow’ are being compared the following difference is noticed (1) Inflorescence size. And (2) Natural season blooming date. (1) Inflorescences of ‘Gold Crest’ are larger than those of ‘Zanmuglow Yellow’. (2) Plants of ‘Gold Crest’ flower earlier in the season than those of ‘Zanmuglow Yellow’.

The following is a description of the plant and characteristics that distinguish ‘Zanmuglow Yellow’ as a new and distinct variety.

The color designations are taken from the plant itself. Accordingly, any discrepancies between the color designations and the colors depicted in the photographs are due to photographic tolerances. The color chart used in this description is: The Royal Horticultural Society Colour Chart, edition 2001.

TABLE 1
Botanical Description of Chrysanthemum × morifolium Ramat.
‘Zanmuglow Yellow’

Bud

| | |
|---|---|
| Size | Small; cross-section 5 mm, |
| | height 3 mm |
| Shape | Round |
| Texture | Pubescent |
| Outside Color | Greyed-green 191A |

Phyllaries

| | |
|---|---|
| Number | 24, arranged in 3 rows |
| Shape | Elliptic |
| Apex | Acute |
| Base | Truncate |
| Margin | Entire |
| Color | Upper side: Green 139A |
| | Lower side: Greyed-green 191A |
| Length and width | 4-5 mm; 1.5-2 mm |
| Texture | Pubescent |

Inflorescence

| | |
|---|---|
| Type | Double |
| Height | 1.5 cm |
| Diameter | 4 cm |
| Peduncle length | 7 cm |
| Peduncle color | Greyed-Green 191B |
| Peduncle diameter | 1.8 mm |
| Peduncle texture | Pubescent |
| Number per branch | Approx 5 inflorescences |
| Duration of flowering | 5 weeks |
| Seeds | Produced in small quantities, ovate, |
| | Greyed-brown 199A, length 1.5 mm, |
| | diameter 0.5 mm |
| Fragrance | Faint chrysanthemum odor |

Color

| | |
|---|---|
| Center of inflorescence | Immature stage: Yellow-Green 144A |
| (disc florets) | Mature stage: Green-Yellow 1B |
| Color of upper surface of the | Yellow 5A |
| ray-florets | |
| Color of the lower surface | Yellow 4B |
| of the ray-florets | |
| Tonality from Distance | A garden mum with yellow flowers |
| Color of the ray-florets after | Yellow 13C |
| aging of the plant | |

Ray florets

| | |
|---|---|
| Texture | Upper and lower surface smooth |
| Number | Ca 170 |
| Shape | Elliptic |
| Apex | Rounded |
| Base | Attenuate |
| Cross-section | Flat |
| Longitudinal axis of | Straight |
| majority | |
| Length of corolla tube | 2 mm |
| Ray-floret margin | Entire |
| Ray-floret length | 1.2-1.8 cm |
| Ray-floret width | 2-6 mm |
| Ratio length / width | Medium |

Disc florets

| | |
|---|---|
| Disc diameter | 1-2 mm |
| Distribution of disc florets | Few, only visible in |
| | mature inflorescence |
| Shape | Tubular |
| Color | Yellow-green 145C at base to |
| | Green Yellow 1B at top |
| Length | 2 mm |
| Diameter | 0.8 mm |

Receptacle

| | |
|---|---|
| Color | Yellow-green 145D |
| Shape | Domed raised |
| Height | 2 mm |
| Diameter | 3 mm |

Reproductive Organs

| | |
|---|---|
| Androecium | Present on only disc florets |
| Stamen length | 3 mm |
| Stamen color | Yellow-green 144A |
| Anther color | Yellow 6D |
| Pollen | Present |
| Pollen color | Yellow 13A |
| Gynoecium | Present on both ray and disc florets |
| Style color | Yellow-green 154C |
| Style Length | 3 mm |
| Stigma color | Yellow 7A |
| Stigma Width | 1 mm |
| Ovary | Enclosed in calyx |

Plant

| | |
|---|---|
| Form | Grown as a spray type pot mum, |
| | outdoor raised and mounded |
| Growth habit | Spherical shape |
| Growth rate | Medium |
| Height | 30 cm |
| Width | 45 cm |
| Stem Color | Greyed-brown 199A |
| Stem Strength | Strong |
| Stem Brittleness | Not brittle |
| Stem Anthocyanin | Not observed |
| Coloration | |
| Internode length | 1-1.5 cm |
| Length of lateral branch | From top to bottom 16 cm |
| Lateral branch color | Green 138A-B |
| Lateral branch, attachment | Medium |
| Lateral branch diameter | 2 mm |
| Branching (average number | Prolific with 8 breaks after |
| of lateral branches) | pinching |
| Natural season blooming | September 22 (week 39) |
| date | |

Foliage

| | |
|---|---|
| Leaf color | Upper side: Green 139A |
| | Lower side: Green 138A |
| Color midvein | Upper side: Yellow-green 147D |
| | Lower side: Yellow-green 148D |
| Size | Small; length 2.5-3.8 cm, |
| | width 1.5-2.8 cm |
| Quantity (number per lateral | Ca. 20 |
| branch) | |
| Shape | Elliptic |
| Texture upper side | Sparsely pubescent |
| Texture under side | Pubescent |
| Venation arrangement | Palmate |
| Shape of the margin | Serrated |
| Shape of Base of Sinus | Rounded |
| Between Lateral Lobes | |
| Margin of Sinus Between | Diverging |
| Lateral Lobes | |
| Shape of Base | Acute to truncate |
| Apex | Mucronulate |
| Petiole length | 2-4 mm |
| Petiole diameter | 2 mm |
| Petiole color | Yellow-green 147D |

TABLE 2
Differences with the comparison variety
(when grown side to side)

| | ‘Zanmuglow Yellow’ | ‘Gold Crest’ |
|---|---|---|
| Inflorescence size | 4 cm | 5 cm |
| Natural season blooming | week 39 | week 37 |
| date | | |

I claim:

1. A new and distinct chrysanthemum plant named ‘Zanmuglow Yellow’ as described and illustrated.
